(19) United States
(12) Patent Application Publication
(10) Pub. No.: US 2024/0197207 A1
(43) Pub. Date: Jun. 20, 2024

(54) Method for Detecting Validity of Human Body Movement

(71) Applicant: FindSatoshi Lab Limited, Tortola (VG)

(72) Inventor: Yan Rong, Hyde Park (AU)

(21) Appl. No.: 18/083,785

(22) Filed: Dec. 19, 2022

(51) Int. Cl. A61B 5/11; A61B 5/02

(52) U.S. Cl. CPC A61B 5/1123; A61B 5/02

(57) ABSTRACT

A method for detecting validity of human body movement is disclosed, comprising: obtaining a user movement dataset comprising a plurality of n-dimensional movement data items corresponding to a human body movement; vectorizing the user movement dataset to obtain a plurality of eigenvectors of the user movement dataset; inputting the plurality of eigenvectors into a validity detection model, wherein the validity detection model comprises a baseline center and a baseline Mahalanobis distance threshold, to determine whether the corresponding human body movement is valid by steps of: determining a proportion, out of the plurality of eigenvectors of the user movement dataset, of eigenvectors having a Mahalanobis distance from the baseline center that does not exceed the baseline Mahalanobis distance threshold; determining whether the proportion is greater than a proportion threshold.

### TECHNICAL FIELD

The present disclosure relates to the field of movement monitoring, and more specifically to a method for detecting the validity of a human body movement.

### BACKGROUND ART

One of the basic daily activities for most people is moving around. In order to encourage people to walk more, a possible way is to monitor human body movements by using smart devices such as wristbands, and provide rewards based on the generated movement records. Typical human body movements include walking, jogging, or running. For example, by jogging outdoors, a user will earn game coins to be used in games or be cashed out for profit. Through this kind of financial gamification (Game-Fi), millions of people can be driven towards a healthier lifestyle and to fight climate change.

However, the commonly used methods for monitoring human body movement today are usually through step counting by mobile devices, GPS path tracking, or a combination of both. For example, one of the commonly used methods is to count the steps via smart wearables. However, this scheme has defects such as poor robustness and susceptibility to being spoofed. For example, Chinese patent CN108332770B discloses a method for preventing cheating in step counting, according to which the technical solution determines whether step counting is a suspected cheating according to the increment of steps in each preset time interval. This scheme is prone to false alarm on the one hand, for example, the user's speed change during running may be misunderstood as suspected cheating; on the other hand, it may not be able to give accurate determination in time for some artificial manipulation of a device for invalid activity recording, for example, using a mechanical device to fix and shake a smart device to simulate walking.

Therefore, there is a great need in the field for a solution that can at least reliably detect the validity of human body movement, so as to determine whether the user has actually performed a valid movement. It should be understood that the technical problems listed above are only examples rather than limitations to the present disclosure, and that the present disclosure is not limited to a technical solution that simultaneously solves all the above technical problems. The technical solutions of the present disclosure can be implemented to solve one or more of the above or other technical problems.

### SUMMARY OF THE INVENTION

The object of the present disclosure is to solve the problem that the existing human body movement monitoring cannot accurately detect whether there are invalid human body movement records. Since a user's movement is exceptionally complex, it is difficult to accurately determine whether there is a fraudulent movement record or other invalid behaviors by a single dimension information, such as step count.

In one aspect of the present disclosure, a method for detecting validity of human body movement is provided, comprising: obtaining a user movement dataset, the user movement dataset comprising a plurality of n-dimensional movement data items corresponding to a human body movement; vectorizing the user movement dataset to obtain a plurality of eigenvectors of the user movement dataset; inputting the plurality of eigenvectors of the user movement dataset into a validity detection model to determine whether the human body movement corresponding to the user movement dataset is valid; wherein the validity detection model comprises a baseline center and a baseline Mahalanobis distance threshold, and wherein determining whether the human body movement corresponding to the user movement dataset is valid comprises: determining a proportion, out of the plurality of eigenvectors of the user movement dataset, of eigenvectors having a Mahalanobis distance from the baseline center that does not exceed the baseline Mahalanobis distance threshold; determining whether the proportion is greater than a proportion threshold; determining that the human body movement corresponding to the user movement dataset is valid in response to the proportion being greater than the proportion threshold; determining the user movement dataset corresponding to human body movement is invalid in response to the proportion being not greater than the proportion threshold.

Preferably, the validity detection model is updated by a training dataset, wherein the training dataset comprises a plurality of n-dimensional movement data items corresponding to a valid human body movement, comprising steps of: vectorizing the training dataset to obtain a plurality of eigenvectors of the training dataset; obtaining the baseline center based on the plurality of eigenvectors of the training dataset, wherein the baseline center is a mean value of the plurality of eigenvectors of the training dataset; determining a Mahalanobis distance for each of the plurality of eigenvectors of the training dataset to the baseline center, and calculating a mean value and a standard deviation of the plurality of Mahalanobis distances, and using a sum of the mean value and the standard deviation as the baseline Mahalanobis distance threshold.

Further preferably, vectorizing one of the user movement dataset and the training dataset comprises: dividing one of the user movement dataset and the training dataset into M data segments, wherein each of the M data segments contains N n-dimensional movement data items; extracting eigenvectors from the 2nd to the Nth movement data items in each of the M data segments, wherein the eigenvector is calculated as a n-dimensional difference between a current movement data item and a first movement data item in each data segment.

Furthermore, dividing one of the user movement dataset and the training dataset into M data segments comprises: applying a sliding window of a length N to smoothly move sequentially from a starting position of one of the training dataset and the user movement dataset to successively acquire the first data item to the Nth data item in one of the user movement dataset and the training dataset as a first data segment, the second data item to the (N+1)th data item as a second data segment, . . . , until the (M−N+1)th data item to the Mth data item as a Mth data segment.

Preferably, each of the plurality of n-dimensional movement data items further comprises at least time information, longitude information, and latitude information. Further preferably, each of the plurality of n-dimensional movement data items further comprises at least one of accumulated steps information, instantaneous movement speed and altitude information. Further preferably, each of the plurality of n-dimensional movement data items further comprises at least one of pulse rate, body temperature, blood oxygen value, and blood pressure value.

Preferably, movement data items are collected at a preset frequency by using at least one sensor in a mobile device to construct the user movement dataset or the training dataset. Preferably, as an alternative, movement data items are collected by invoking at least one of a GPS positioning service, an application program, a gyroscope of a smart device, or a combination thereof, to construct one of the training dataset and the user movement dataset.

Preferably, the user movement dataset is divided into a plurality of subsets, such that a change rate in at least one of preset dimensions of the plurality of n-dimensional movement data items in each of the plurality of subsets does not exceed a preset value, and determining for each of the plurality of subsets whether a human body movement of the corresponding subset is valid.

Preferably, in response to a user's indication to pause a movement, movement data items collected during a pause period are tagged, and further the tagged movement data items are removed when the user's movement dataset is obtained.

Preferably, the validity detection model further comprises an abnormal baseline center and an abnormal Mahalanobis distance threshold, wherein the abnormal baseline center and the abnormal Mahalanobis distance threshold are updated by an abnormal training dataset; wherein the abnormal training dataset comprises a plurality of movement data items corresponding to invalid human body movement behavior, the invalid human body movement behavior comprising at least one of: movement by vehicle, movement with an aid of a pedometer, movement by pet, GPS signal and step count spoofing using a smart device simulator, and falsified movement data using a script; the method further comprises: in response to that the human body movement corresponding to the user movement dataset is determined to be invalid, further determining the proportion, out of the plurality of eigenvectors of the user movement dataset, of eigenvectors having a Mahalanobis distance from the abnormal baseline center that does not exceed the abnormal Mahalanobis distance threshold; determining whether the proportion is greater than a proportion threshold; determining that the human body movement corresponding to the user movement dataset is an invalid human body movement behavior corresponding to the abnormal training dataset in response to the proportion being greater than a proportion threshold.

Preferably, the validity detection model is deployed on a device terminal with edge computing capability, and the method is executed by the device terminal.

In a second aspect of the present disclosure, a non-transitory computer-readable medium having storage content is further provided, wherein the storage content causes a computing system to perform automated operations, comprising: obtaining a user movement dataset, the user movement dataset comprising a plurality of n-dimensional movement data items corresponding to a human body movement; vectorizing the user movement dataset to obtain a plurality of eigenvectors of the user movement dataset; inputting the plurality of eigenvectors of the user movement dataset into a validity detection model to determine whether the human body movement corresponding to the user movement dataset is valid; wherein the validity detection model comprises a baseline center and a baseline Mahalanobis distance threshold, and wherein determining whether the human body movement corresponding to the user movement dataset is valid comprises; determining a proportion, out of the plurality of eigenvectors of the user movement dataset, of eigenvectors having a Mahalanobis distance from the baseline center that does not exceed the baseline Mahalanobis distance threshold; determining whether the proportion is greater than a proportion threshold; determining that the human body movement corresponding to the user movement dataset is valid in response to the proportion being greater than the proportion threshold; determining that the human body movement corresponding to the user movement dataset is invalid in response to the proportion being not greater than the proportion threshold.

In a third aspect of the present disclosure, a system is further provided, comprising: one or more processors; a wireless communication module configured to obtain a user movement dataset; and at least one memory having stored instructions that, when executed by at least one of the one or more processors, cause the system to perform automated operations, comprising: obtaining a user movement dataset, the user movement dataset comprising a plurality of n-dimensional movement data items corresponding to human body movement; vectorizing the user movement dataset to obtain a plurality of eigenvectors of the user movement dataset; inputting the plurality of eigenvectors of the user movement dataset into a validity detection model to determine whether the human body movement corresponding to the user movement dataset is valid; wherein the validity detection model comprises a baseline center and a baseline Mahalanobis distance threshold, and wherein determining whether the human body movement corresponding to the user movement dataset is valid comprises: determining a proportion, out of the plurality of eigenvectors of the user movement dataset, of eigenvectors having a Mahalanobis distance from the baseline center that does not exceed the baseline Mahalanobis distance threshold; determining whether the proportion is greater than a proportion threshold; determining that the human body movement corresponding to the user movement dataset is valid in response to the proportion being greater than the proportion threshold; determining that the human body movement corresponding to the user movement dataset is invalid in response to the proportion being not greater than the proportion threshold.

On one hand, according to the solution of the present disclosure, the validity detection model is trained using a large amount of training datasets, which comprise data from sensors, GPS tracking, other personal health information such as blood pressure, etc. On the other hand, the user movement dataset is processed by vectorization, and then input into the validity detection model to determine whether a Mahalanobis distance between each of the obtained eigenvectors and the baseline center exceeds a threshold. Therefore, the solution according to the present disclosure has high scalability, while at the same time allowing comprehensive collection of motion-related data items to serve as a data basis. By assessing whether the user movement dataset is invalid or not, and further identifying any violation behavior that lead to invalidation, the accuracy rate of identifying invalid behaviors can be improved while reducing the false alarm rate.

### BRIEF DESCRIPTION OF THE DRAWINGS

FIG. 1 illustrates a schematic flow diagram of a method for detecting the validity of human body movement according to an embodiment of the present disclosure;

FIG. 2 illustrates a schematic flow chart of a method for detecting the validity of human body movement according to another embodiment of the present disclosure;

FIG. 3 illustrates a flow chart of an embodiment of a method for detecting the validity of human body movement according to the present disclosure;

FIG. 4 illustrates a schematic diagram of an embodiment of a method for vectorization processing according to the present disclosure;

FIG. 5 illustrates a schematic diagram of an embodiment of a method for extracting eigenvectors according to the present disclosure;

FIG. 6 illustrates a flow chart of an embodiment of a method for updating a validity detection model by a training dataset according to the present disclosure; and

FIG. 7 illustrates a schematic diagram of a system according to an embodiment of the present disclosure.

### DESCRIPTION OF THE PREFERRED EMBODIMENTS

The method of the present disclosure will be described in detail below with reference to the accompanying drawings and specific embodiments. It should be understood that the embodiments shown in the accompanying drawings and described below are merely illustrative and are not intended to be a limitation of the present disclosure.

Users may carry mobile devices such as smartphones or wearables with them when they are on the move, such as walking, jogging, cycling, and running. Such mobile devices are usually configured with various types of sensors, such as accelerometers, gyroscopes, magnetometers, etc., which can be used to obtain information about the user's movement, such as step counts, movement speed, etc.; and configured with distance monitoring components, such as GPS module, which can be used to measure the user's movement in real time, such as moving direction, moving distance, longitude and latitude information, etc. At the end of the movement, the user can choose to stop recording the corresponding user movement data, and various types of movement data information generated during the movement can be aggregated for further recording or analysis.

To encourage people to move around in the real world, people's movements can be tracked and rewarded, for example, users earn rewards for their movements, wherein such rewards can comprise but are not limited to gift cards, vouchers, virtual currency, cryptocurrencies such as tokens or coins, etc.

However, some participants may try to cheat to get more rewards. Such cheating behavior can affect the confidence and motivation of other participants. Therefore, in order to ensure fairness, the validity of such movement data needs to be assessed to flag invalid movement records generated by any cheating behavior.

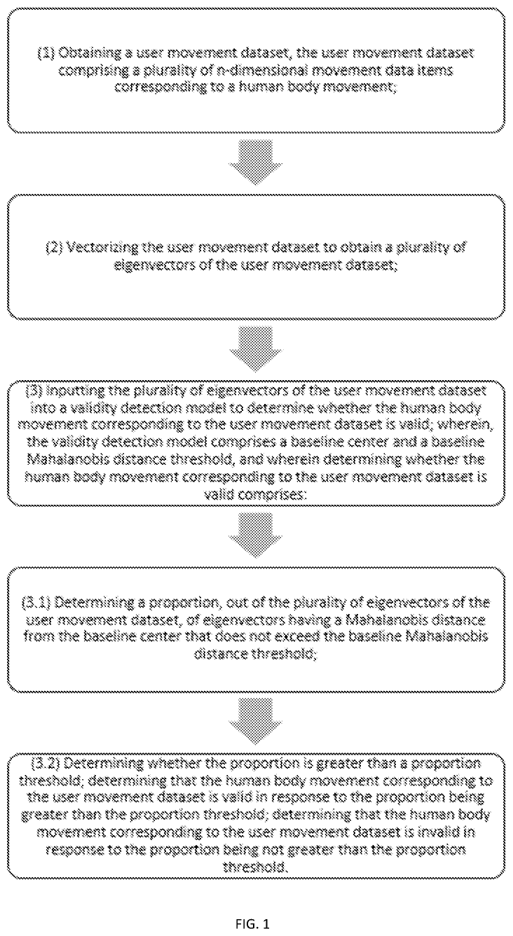

In a first aspect, a method for detecting validity of human body movement is disclosed, as shown in FIG. 1, comprising steps of:
(1) obtaining a user movement dataset, the user movement dataset comprising a plurality of n-dimensional movement data items corresponding to a human body movement;
(2) vectorizing the user movement dataset to obtain a plurality of eigenvectors of the user movement dataset;
(3) inputting the plurality of eigenvectors of the user movement dataset into a validity detection model to determine whether the human body movement corresponding to the user movement dataset is valid; wherein the validity detection model comprises a baseline center and a baseline Mahalanobis distance threshold, and wherein determining whether the human body movement corresponding to the user movement dataset is valid comprises:
(3.1) determining a proportion, out of the plurality of eigenvectors of the user movement dataset, of eigenvectors having a Mahalanobis distance from the baseline center that does not exceed the baseline Mahalanobis distance threshold;
(3.2) determining whether the proportion is greater than a proportion threshold; determining that the human body movement corresponding to the user movement dataset is valid in response to the proportion being greater than the proportion threshold; determining that the human body movement corresponding to the user movement dataset is invalid in response to the proportion being not greater than the proportion threshold.

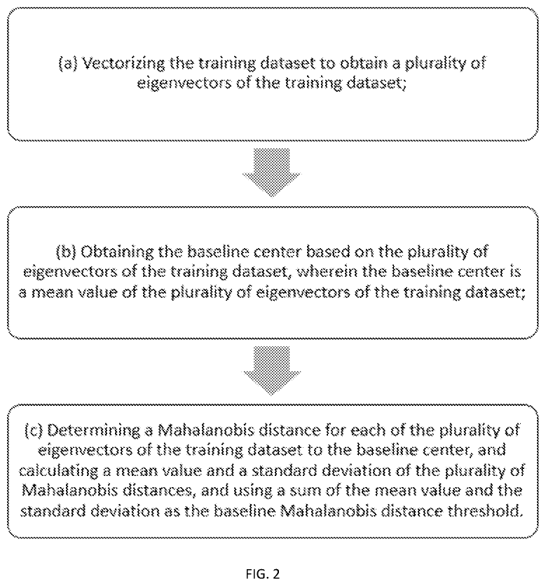

The method for detecting validity of human body movement according to the present disclosure, as shown in FIG. 2, further comprises: updating the validity detection model by a training dataset, wherein the training dataset comprises a plurality of n-dimensional movement data items corresponding to a valid human body movement, comprising steps of:
(a) vectorizing the training dataset to obtain a plurality of eigenvectors of the training dataset;
(b) obtaining the baseline center based on the plurality of eigenvectors of the training dataset, wherein the baseline center is a mean value of the plurality of eigenvectors of the training dataset;
(c) determining a Mahalanobis distance for each of the plurality of eigenvectors of the training dataset to the baseline center, and calculating a mean value and a standard deviation of the plurality of Mahalanobis distances, and using a sum of the mean value and the standard deviation as the baseline Mahalanobis distance threshold.

The above methods are general exemplary embodiments of the method for detecting validity of human body movement, and the steps will be described in more detail below.

### Detecting Validity by a Validity Detection Model

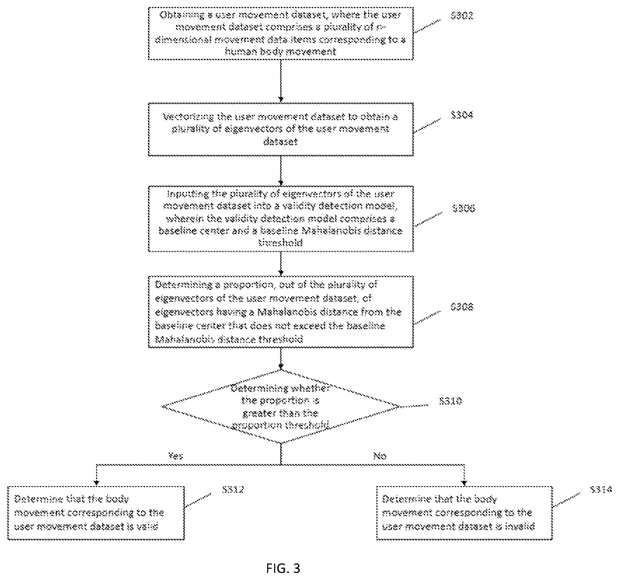

FIG. 3 illustrates a flowchart of an embodiment of a method for detecting the validity of human body movement in accordance with the present disclosure. As shown in FIG. 3, the method 300 comprises a plurality of steps:

In step S302, a user movement dataset is obtained, and the user movement dataset comprises a plurality of n-dimensional movement data items corresponding to a human body movement. For example, a movement data item corresponding to a respective human body movement is collected at a preset frequency by using at least one sensor in a mobile device to construct the user movement dataset. Specifically, for example, movement information is collected as an n-dimensional movement data item every 1 second by the GPS module of a mobile device (such as a smart phone or a sports bracelet). Each n-dimensional movement data item collected can comprise at least one of: time information, GPS location information (such as longitude, latitude, altitude), accumulated steps or instantaneous movement speed, etc. In addition, in some embodiments, the n-dimensional movement data item can also comprise at least one of: pulse rate, body temperature, blood oxygen value, blood pressure value, etc. Alternatively, as another embodiment, movement data items corresponding to a respective human body movement can be collected by invoking at least one of: a GPS positioning service, an application program, a gyroscope of the smart device, or a combination thereof, to construct one of the user movement dataset and the training dataset. Preferably, each of the plurality of n-dimensional movement data items further comprises at least time information, longitude information, and latitude information. Further preferably, each of the plurality of n-dimensional movement data items further comprises at least one of accumulated steps information, instantaneous movement speed and altitude information.

Preferably, in order to further reduce the amount of resources consumed in executing the method according to this embodiment, and reduce unnecessary triggers to execute the method according to this embodiment, after obtaining the user movement dataset, preprocessing can be performed to confirm whether there is an abnormality in the GPS positioning, or whether there is no movement according to the GPS positioning information, or whether the GPS information cannot be obtained or the GPS information is disturbed violently, or whether the step counts in the user movement dataset remains unchanged, or whether the speed in the user movement dataset is always 0, and any other abnormalities etc. If any of the above-mentioned abnormalities occur, it is directly determined that the human body movement corresponding to the user movement dataset is invalid.

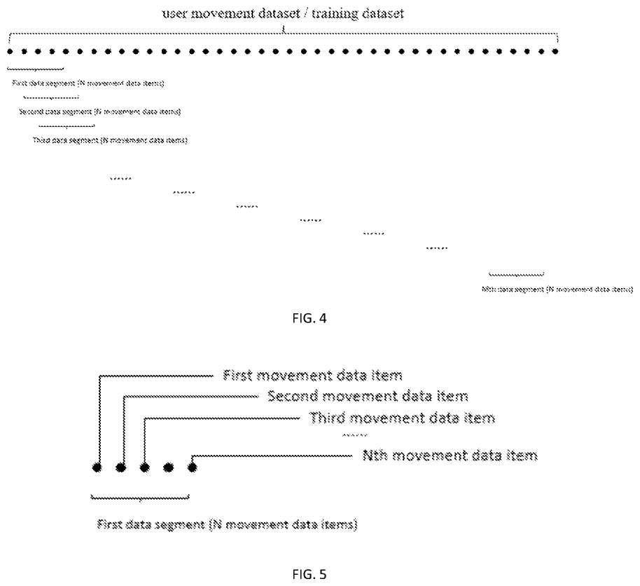

In step S304, the user movement dataset is vectorized to obtain a plurality of eigenvectors of the user movement dataset. Specifically, for example, as shown in FIG. 4, vectorizing one of the user movement dataset and the training dataset comprises: dividing one of the user movement dataset and the training dataset into M data segments, wherein each of the M data segments contains N n-dimensional movement data items; further, extracting eigenvectors from the 2nd to the Nth movement data items in each of the M data segments, wherein the eigenvector is calculated as a n-dimensional difference between a current movement data item and a first movement data item in each data segment. Here, dividing one of the user movement dataset and the training dataset into M data segments comprises: applying a sliding window of a length N to smoothly move sequentially from a starting position of the training dataset or the user movement dataset to successively acquire the first data item to the Nth data item in one of the user movement dataset and the training dataset as a first data segment, the second data item to the (N+1)th data item as a second data segment, . . . , until the (M−N+1)th data item to the Mth data item as a Mth data segment. Specifically, as shown in FIG. 5, taking the first data segment as an example, it shows the first movement data item, the second movement data item, and the Nth movement data item in the data segment, where the difference between the second movement data item and the first movement data item in n dimensions is obtained as a eigenvector v₁⁽¹⁾, the difference between the third movement data item and the first movement data item in n dimensions is obtained as a eigenvector v₁⁽²⁾, and so on, until the difference between the Nth movement data item and the first movement data item in n dimensions is obtained as a eigenvector vv₁^{(N-1)}, thus obtaining N−1 eigenvectors, where each eigenvector is an n-dimensional vector. For M data fragments which each contains N n-dimensional data items, a total of M*(N−1) eigenvectors are extracted.

In step S306, the plurality of eigenvectors of the user movement dataset are input into a validity detection model, wherein the validity detection model comprises a baseline center and a baseline Mahalanobis distance threshold. For example, the baseline center and the baseline Mahalanobis distance threshold can be predetermined in the validity detection model.

In step S308, a proportion, out of the plurality of eigenvectors of the user movement dataset, of eigenvectors having a Mahalanobis distance from the baseline center that does not exceed the baseline Mahalanobis distance threshold is determined. Specifically, for example, the b-th eigenvector obtained from the a-th 20) data segment is denoted as vₐ^{(b)}, the baseline center is denoted as u, and the baseline Mahalanobis distance threshold is denoted as dₛ, then the Mahalanobis distance between the eigenvector and the baseline center d (vₐ^{(b)}) can be obtained by the following formula:

$$d ( {v}_{a}^{( b )} ) = \sqrt{{( {v}_{a}^{( b )} - μ )}^{T} {Σ}^{- 1} ( {v}_{a}^{( b )} - μ )} ,$$

where Σ denotes the covariance matrix of all the eigenvectors obtained from the user movement dataset; and it is further determined whether the Mahalanobis distance between the eigenvector and the baseline center d (vₐ^{(b)}) is less than or equal to the baseline Mahalanobis distance threshold dₛ. Finally, whether the proportion is greater than a proportion threshold is determined. More specifically, the human body movement corresponding to the user movement dataset is determined as valid in response to the proportion being greater than the proportion threshold is determined; and the human body movement corresponding to the user movement dataset is determined as invalid in response to the proportion being not greater than the proportion threshold.

It should be understood that since the movement data items in the collected user movement dataset for a user's exercising often differ significantly in some dimensions, such as instantaneous movement speed, it is therefore necessary to reduce the sensitivity to changes in any individual dimensions when detecting the validity in order to reduce the false positive rate. The technical solution of the present disclosure uses the Mahalanobis distance as the basis for validity detection, which can bring the above-mentioned obvious benefits, reduce the false alarm rate, and improve the accuracy of determination.

In step S310, whether the proportion is greater than the proportion threshold is determined. In response to the proportion being greater than the proportion threshold, step S312 is executed to determine that the body movement corresponding to the user movement dataset is valid; in response to the proportion being not greater than the proportion threshold, step S314 is executed to determine that the body movement corresponding to the user movement dataset is invalid. As an example, the proportion threshold can be preset in the validity detection model. As a further optimization, different proportion thresholds can be set according to the application scenario selected by the user, such as jogging or walking.

In some embodiments, the user movement dataset can be divided into a plurality of subsets, such that a change rate in at least one of preset dimensions of the plurality of n-dimensional movement data items in each of the plurality of subsets does not exceed a preset value. Each of the plurality of subsets is detected to determine whether a human body movement of the corresponding subset is valid. The actual movement of the user can be more complicated. For example, the user can walk for a portion of the distance and run or cycle for another portion of the distance in the movement. For such situations, according to the method of the present disclosure, the collected user movement dataset can be divided into subsets depending on whether the movement speed or any other dimension has a sudden change, and each of the divided subsets can be evaluated to verify its validity, so as to obtain more objective detection results.

In some embodiments, in response to a user's indication that the movement is paused, the movement data items collected during the pause can be tagged, and the tagged movement data items collected during the pause can be removed when obtaining the user movement dataset. The user may experience subjective or non-subjective pauses in motion during movement, which can adversely affect the effectiveness of validity detection if the movement data items during the pause period continue to be collected and aggregated into the user movement dataset. In some cases, the mobile device can be instructed to pause the collection of movement data items during the pause in a movement, or alternatively, to tag the movement data items collected during the pause and remove the tagged movement data items from the user movement dataset during preprocessing. For example, the user can issue an instruction to pause movement via the mobile device, and then issue an instruction to resume movement after a period of time. The mobile data items collected during the pause based on the time stamps corresponding to the instruction to pause the movement and the instruction to resume the movement are removed from the user motion dataset.

### Updating the Validity Detection Model by a Training Dataset

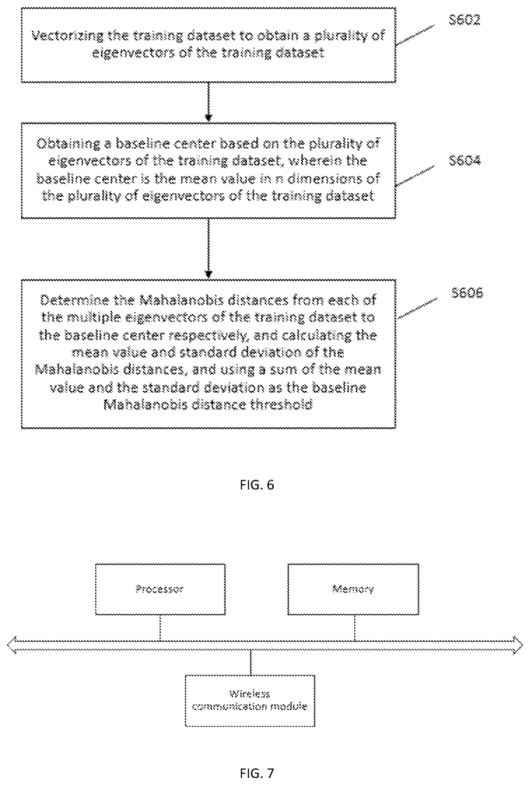

FIG. 6 illustrates a flowchart of an embodiment of a method for updating a validity detection model by a training dataset according to the present disclosure. A single training dataset contains a plurality of n-dimensional movement data items corresponding to a valid human body movement. As shown in FIG. 6, the method 600 comprises a plurality of steps:

In step S602, the training dataset is vectorized to obtain a plurality of eigenvectors of the training dataset. Here, the vectorization processing of the training dataset is performed in a similar manner to the vectorization processing applied to the user movement dataset in step S104 as described above. Specifically, the vectorization processing can comprise steps of: dividing the training dataset into a plurality of data segments, where each of the data segments contains N n-dimensional movement data items. Here, dividing the training dataset into M data segments comprises: applying a sliding window of a length N to smoothly move sequentially from a starting position of the training dataset or the user movement dataset to successively acquire the first data item to the Nth data item in one of the user movement dataset and the training dataset as a first data segment, the second data item to the (N+1)th data item as a second data segment, . . . , until the (M−N+1)th data item to the Mth data item as a Mth data segment. Further, eigenvectors are extracted for the 2nd to Nth movement data items in each data segment respectively, where the eigenvector extracted for each movement data item is a difference in n dimensions between the current movement data item and the first movement data item in the data segment.

In step S604, a baseline center is obtained based on the plurality of eigenvectors of the training dataset obtained in step S602, wherein the baseline center is the mean value in n dimensions of the plurality of eigenvectors of the training dataset.

In step S606, the Mahalanobis distances from each of the multiple eigenvectors of the training dataset to the baseline center are respectively determined, and the mean value and standard deviation of the Mahalanobis distances are calculated, and a sum of the mean value and the standard deviation is used as the baseline Mahalanobis distance threshold.

As a result, the validity detection model, in particular the baseline center and the baseline Mahalanobis distance threshold in the validity detection model, are updated.

In some embodiments, a large number of human body movements that have been validated as valid are tracked to obtain a training dataset, wherein the n-dimensional movement data items in the training dataset conform to a natural distribution law, such as a normal distribution, in terms of movement speed, moving mode, etc.

In some optimized embodiments, the training dataset comprises a movement dataset corresponding to the user's past human body movements that have been validated as valid. By taking the movement dataset corresponding to the user's past valid human body movement as a training dataset, it can be iterated continuously to achieve a gradual convergence of the baseline Mahalanobis distance threshold, and finally achieve a more accurate determination for a single user.

### Identifying Invalid Behaviors

In some embodiments, the validity detection model further comprises an abnormal baseline center and an abnormal Mahalanobis distance threshold, wherein the abnormal baseline center and the abnormal Mahalanobis distance threshold are updated by an abnormal training dataset; wherein the abnormal training dataset comprises a plurality of movement data items corresponding to an invalid human body movement behavior, the invalid human body movement behavior comprising at least one of: movement by vehicle, movement with an aid of a pedometer, movement by pet, GPS signal and step count spoofing using a smart device simulator, and falsified movement data using a script. Movement data of various types of invalid human body movement behaviors are collected and used to construct abnormal training datasets.

Based on the abnormal baseline center and the abnormal Mahalanobis distance threshold, the user motion dataset corresponding to the human movement that has been determined to be invalid can be further identified to belong to a specific type of invalid human body movement behavior. Specifically, the method further comprises: in response to that the human body movement corresponding to the user movement dataset is determined to be invalid, further determining the proportion, out of the plurality of eigenvectors of the user movement dataset, of eigenvectors having a Mahalanobis distance from the abnormal baseline center that does not exceed the abnormal Mahalanobis distance threshold; determining whether the proportion is greater than a proportion threshold; determining that the human body movement corresponding to the user movement dataset is an invalid human body movement behavior corresponding to the abnormal training dataset in response to the proportion being greater than a proportion threshold.

In some embodiments, a plurality of abnormal training datasets may be comprised, wherein each abnormal training dataset corresponds to one invalid human body movement behavior. The above determination method is performed on the user movement dataset and a plurality of abnormal training datasets respectively, to determine an abnormal training dataset that is closest to the eigenvector of the user movement dataset, to which the corresponding invalid human body movement behavior is identified as the invalid cause corresponding to the user movement dataset.

### Deploying the Validity Detection Model

In some embodiments, the validity detection model can be deployed on a remote server, where the user movement dataset is uploaded to the server to execute the validity detection method.

In some other embodiments, the validity detection model can be deployed on a device terminal with edge computing capability, where the user movement dataset is stored locally, and the validity detection method is executed by the device terminal.

It is understandable that the term “server” may not necessarily be in the form of a single physical machine, but may also be, for example, a distributed or cloud computing service, engine, or platform.

In a second aspect, a non-transitory computer-readable medium is disclosed, which has stored content that causes a computing system to perform automated operations, comprising: obtaining a user movement dataset, the user movement dataset comprising a plurality of n-dimensional movement data items corresponding to a human body movement; vectorizing the user movement dataset to obtain a plurality of eigenvectors of the user movement dataset; inputting the plurality of eigenvectors of the user movement dataset into a validity detection model to determine whether the human body movement corresponding to the user movement dataset is valid; wherein the validity detection model comprises a baseline center and a baseline Mahalanobis distance threshold.

Here, determining whether the human body movement corresponding to the user movement dataset is valid comprises: determining a proportion, out of the plurality of eigenvectors of the user movement dataset, of eigenvectors having a Mahalanobis distance from the baseline center that does not exceed the baseline Mahalanobis distance threshold; determining whether the proportion is greater than a proportion threshold; determining that the human body movement corresponding to the user movement dataset is valid in response to the proportion being greater than the proportion threshold; determining that the human body movement corresponding to the user movement dataset is invalid in response to the proportion being not greater than the proportion threshold.

In a third aspect, as shown in FIG. 7, a system is disclosed, comprising: one or more processors; a wireless communication module configured to obtain a user movement dataset; and at least one memory having stored instructions that, when executed by at least one of the one or more processors, cause the system to perform automated operations, comprising: obtaining a user movement dataset, the user movement dataset comprising a plurality of n-dimensional movement data items corresponding to human body movement; vectorizing the user movement dataset to obtain a plurality of eigenvectors of the user movement dataset; inputting the plurality of eigenvectors of the user movement dataset into a validity detection model to determine whether the human body movement corresponding to the user movement dataset is valid; wherein the validity detection model comprises a baseline center and a baseline Mahalanobis distance threshold. Here, determining whether the human body movement corresponding to the user movement dataset is valid comprises: determining a proportion, out of the plurality of eigenvectors of the user movement dataset, of eigenvectors having a Mahalanobis distance from the baseline center that does not exceed the baseline Mahalanobis distance threshold; determining whether the proportion is greater than a proportion threshold; determining that the human body movement corresponding to the user movement dataset is valid in response to the proportion being greater than the proportion threshold; determining that the human body movement corresponding to the user movement dataset is invalid in response to the proportion being not greater than the proportion threshold.

In summary, the method of the present disclosure can improve the accuracy of detection while reducing the false alarm rate and ensuring the efficiency of execution by training the validity detection model using a large number of training data sets, processing them by vectorization, and inputting the user motion data set into the validity detection model to determine whether the Mahalanobis distance between the obtained eigenvector of the user movement dataset and the baseline center exceeds a threshold value.

While various embodiments of aspects of the disclosure have been described for the purposes of the present disclosure, it should not be construed that the teachings of the present disclosure are limited to these embodiments. The features disclosed in a specific embodiment are not limited to the embodiment, but can be combined with features disclosed in different embodiments. For example, one or more features and/or operations of the method according to the present disclosure described in one embodiment may also be applied individually, in combination or as a whole in another embodiment. Furthermore, it should be understood that the method steps described above can be performed sequentially, in parallel, combined into fewer steps, split into more steps, combined and/or omitted in a manner other than described. It should be understood by those skilled in the art that further optional embodiments and variants are possible, and that various changes and modifications can be made to the method steps described above without departing from the scope defined by the claims of the present disclosure.

1. A method for detecting validity of human body movement, the method comprising steps of:
obtaining a user movement dataset, the user movement dataset comprising a plurality of n-dimensional movement data items corresponding to a human body movement;
vectorizing the user movement dataset to obtain a plurality of eigenvectors of the user movement dataset; and
inputting the plurality of eigenvectors of the user movement dataset into a validity detection model to determine whether the human body movement corresponding to the user movement dataset is valid; wherein the validity detection model comprises a baseline center and a baseline Mahalanobis distance threshold, and wherein determining whether the human body movement corresponding to the user movement dataset is valid comprises:
determining a proportion, out of the plurality of eigenvectors of the user movement dataset, of eigenvectors having a Mahalanobis distance from the baseline center that does not exceed the baseline Mahalanobis distance threshold; and
determining whether the proportion is greater than a proportion threshold; determining that the human body movement corresponding to the user movement dataset is valid in response to the proportion being greater than the proportion threshold; determining that the human body movement corresponding to the user movement dataset is invalid in response to the proportion being not greater than the proportion threshold.

2. The method according to claim 1, further comprising a step of updating the validity detection model by a training dataset, wherein the training dataset comprises a plurality of n-dimensional movement data items corresponding to valid human body movement, which comprises steps of:
vectorizing the training dataset to obtain a plurality of eigenvectors of the training dataset;
obtaining the baseline center based on the plurality of eigenvectors of the training dataset, wherein the baseline center is a mean value of the plurality of eigenvectors of the training dataset; and
determining a Mahalanobis distance for each of the plurality of eigenvectors of the training dataset to the baseline center, and calculating a mean value and a standard deviation of the plurality of Mahalanobis distances, and using a sum of the mean value and the standard deviation as the baseline Mahalanobis distance threshold.

3. The method according to claim 2, wherein vectorizing one of the user movement dataset and the training dataset comprises:
dividing one of the user movement dataset and the training dataset into M data segments, wherein each of the M data segments contains N n-dimensional movement data items; and
extracting eigenvectors from the 2nd to the Nth movement data items in each of the M data segments, wherein the eigenvector is calculated as a n-dimensional difference between a current movement data item and a first movement data item in each data segment.

4. The method according to claim 3, wherein dividing one of the user movement dataset and the training dataset into M data segments comprises:
applying a sliding window of a length N to smoothly move sequentially from a starting position of one of the training dataset and the user movement dataset to successively acquire the first data item to the Nth data item in one of the user movement dataset and the training dataset as a first data segment, the second data item to the (N+1)th data item as a second data segment, and so on until the (M−N+1)th data item to the Mth data item as a Mth data segment.

5. The method according to claim 1, wherein each of the plurality of n-dimensional movement data items comprises at least time information, longitude information, and latitude information.

6. The method according to claim 5, wherein each of the plurality of n-dimensional movement data items further comprises at least one of accumulated steps information, instantaneous movement speed, and altitude information.

7. The method according to claim 5, wherein each of the plurality of n-dimensional movement data items further comprises at least one of pulse rate, body temperature, blood oxygen value, and blood pressure value.

8. The method according to claim 2, further comprising a step of collecting movement data items at a preset frequency by using at least one sensor in a mobile device to construct the user movement dataset or the training dataset.

9. The method according to claim 2, further comprising a step of
collecting movement data items by invoking at least one of a GPS positioning service, an application program, a gyroscope of a smart device, or a combination thereof, to construct one of the training dataset and the user movement dataset.

10. The method according to claim 1, further comprising:
dividing the user movement dataset into a plurality of subsets, such that a change rate in at least one of preset dimensions of the plurality of n-dimensional movement data items in each of the plurality of subsets does not exceed a preset value, and determining for each of the plurality of subsets whether a human body movement of the corresponding subset is valid.

11. The method according to claim 1, further comprising:
tagging movement data items collected during a pause period in response to a user's indication to pause a movement, and removing the tagged movement data items when obtaining the user movement dataset.

12. The method according to claim 1, wherein the validity detection model further comprises an abnormal baseline center and an abnormal Mahalanobis distance threshold, wherein the abnormal baseline center and the abnormal Mahalanobis distance threshold are updated by an abnormal training dataset; wherein the abnormal training dataset comprises a plurality of movement data items corresponding to an invalid human body movement behavior, the invalid human body movement behavior comprises at least one of: movement by vehicle, movement with an aid of a pedometer, movement by pet, GPS signal and step count spoofing using a smart device simulator, and falsified movement data using a script;
the method further comprises:
in response to that the human body movement corresponding to the user movement dataset is determined to be invalid, further determining the proportion, out of the plurality of eigenvectors of the user movement dataset, of eigenvectors having a Mahalanobis distance from the abnormal baseline center that does not exceed the abnormal Mahalanobis distance threshold; and
determining whether the proportion is greater than a proportion threshold; determining that the human body movement corresponding to the user movement dataset is an invalid human body movement behavior corresponding to the abnormal training dataset in response to the proportion being greater than a proportion threshold.

13. The method according to claim 1, further comprising,
the validity detection model is deployed on a device terminal with edge computing capability, and the method is executed by the device terminal.

14. A non-transitory computer-readable medium having storage content, wherein the storage content causes a computing system to perform automated operations, comprising:
obtaining a user movement dataset, the user movement dataset comprising a plurality of n-dimensional movement data items corresponding to a human body movement;
vectorizing the user movement dataset to obtain a plurality of eigenvectors of the user movement dataset; and
inputting the plurality of eigenvectors of the user movement dataset into a validity detection model to determine whether the human body movement corresponding to the user movement dataset is valid; wherein the validity detection model comprises a baseline center and a baseline Mahalanobis distance threshold, and wherein determining whether the human body movement corresponding to the user movement dataset is valid comprises:
determining a proportion, out of the plurality of eigenvectors of the user movement dataset, of eigenvectors having a Mahalanobis distance from the baseline center that does not exceed the baseline Mahalanobis distance threshold; and
determining whether the proportion is greater than a proportion threshold; determining that the human body movement corresponding to the user movement dataset is valid in response to the proportion being greater than the proportion threshold; determining that the human body movement corresponding to the user movement dataset is invalid in response to the proportion being not greater than the proportion threshold.

15. A system, comprising:
one or more processors;
a wireless communication module configured to obtain a user movement dataset;
and at least one memory having stored instructions that, when executed by at least one of the one or more processors, cause the system to perform automated operations comprising:
obtaining a user movement dataset, the user movement dataset comprising a plurality of n-dimensional movement data items corresponding to human body movement;
vectorizing the user movement dataset to obtain a plurality of eigenvectors of the user movement dataset; and
inputting the plurality of eigenvectors of the user movement dataset into a validity detection model to determine whether the human body movement corresponding to the user movement dataset is valid; wherein the validity detection model comprises a baseline center and a baseline Mahalanobis distance threshold, and wherein determining whether the human body movement corresponding to the user movement dataset is valid comprises:
determining a proportion, out of the plurality of eigenvectors of the user movement dataset, of eigenvectors having a Mahalanobis distance from the baseline center that does not exceed the baseline Mahalanobis distance threshold; and
determining whether the proportion is greater than a proportion threshold; determining that the human body movement corresponding to the user movement dataset is valid in response to the proportion being greater than the proportion threshold; determining that the human body movement corresponding to the user movement dataset is invalid in response to the proportion being not greater than the proportion threshold.
